(19) United States
(12) Patent Application Publication
(10) Pub. No.: US 2005/0143803 A1
(43) Pub. Date: Jun. 30, 2005

(54) Protective sheath for drug coated stent

(75) Inventors: James R. Watson, Santa Rosa, CA (US); David Doty, Forestville, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/993,341

(22) Filed: Nov. 19, 2004

Related U.S. Application Data
(60) Provisional application No. 60/532,797, filed on Dec. 24, 2003.

(51) Int. Cl. A61F 2/06

(52) U.S. Cl. 623/1.11

(57) ABSTRACT

The present invention provides a system for packaging a drug coated stent, including a transport package body including an arcuate catheter guide and a catheter. The catheter has a drug coated stent disposed thereon to be received in the catheter guide. A sheath including at least one reduced thickness region therein to increase flexibility of the sheath is disposed on the catheter, which encloses the drug coated stent.

### RELATED APPLICATIONS

This application claims the benefit of U.S. Provisional Patent Application 60/532,797 filed Dec. 24, 2003.

### FIELD OF THE INVENTION

This invention relates generally to protective sheaths for biomedical stents. More specifically, the invention relates to a protective sheath for biomedical drug coated stents to be used in packaging for shipping.

### BACKGROUND OF THE INVENTION

Drug coated stents can improve the overall effectiveness of angioplasty and stenotic procedures performed on the cardiovascular system and other vessels within the body by delivering potent therapeutic compounds at the point of infarction. Drugs such as anti-inflammatants and anti-thrombogenics may be dispersed within the drug-polymer coating and released after insertion and deployment of the stent. These drugs and coatings can reduce the trauma to the local tissue bed, aid in the healing process, and significantly reduce the narrowing or constriction of the blood vessel that can reoccur where the stent is placed.

However, if the drug coated stents are packaged to ship to a medical practitioner, without a protective sheath, some of the drug on the stent may rub off of the stent while it is in the package. In a majority of cases, the drug coated stent attached to a catheter is covered with a protective sleeve and then inserted inside a hollow shipping tube. The shipping tube is then configured into a hoop to fit within a shipping box. During the bending of the hoop some of the drug may rub off into the protective sleeve as the stent and sleeve contact the side of the hoop.

In other cases, the drug coated stent attached to a catheter is placed within a protective sleeve and then pushed through the length of the looped shipping hoop, so that at least one point of the stent rubs against an inner surface of the hollow tube and the drug on the stent is removed from that point by friction. If the catheter is twisted upon insertion into the hollow tube, multiple points on the stent may have the drug removed by friction.

Sometimes the drug coated stent attached to a catheter is placed in a protective cover. The catheter with attached stent is then wrapped into a plurality of loops and connected to a tray. The stent may rub against the tray during the process of shipping on at least one point and the drug may be removed from that point by friction between the stent and the tray.

In all these packaging systems the protective sleeve is not always sufficient to prevent a quantity of drug from being rubbed off the stent. In a case when the drug is rubbed off, the drug intended for release to aid a patient is then reduced by an unknown and variable amount. This will result in variable aid for the patient with variable healing effects. It is desirable to package drug coated stents in a manner which prevents any reduction in the amount of drug on the stent.

### SUMMARY OF THE INVENTION

It is an object of this invention to provide a sheath to be used in a packaging system to prevent friction on a drug coated stent from removing the drug applied on the stent by overcoming the deficiencies and limitations described above.

One aspect of the present invention provides a system for packaging a drug coated stent, including a transport package body with an arcuate catheter guide. The system also includes a catheter attached to the drug coated stent and received in the catheter guide. A sheath is disposed on the catheter and encloses the drug coated stent. The sheath includes at least one reduced thickness region to increase flexibility of the sheath.

A second aspect of the present invention provides a sheath for protecting a drug coated stent, including a body portion including a central lumen for receiving a drug coated stent. The body portion includes at least one reduced thickness region to increase flexibility of the sheath.

A third aspect of this invention provides a method for packaging a catheter mounted drug coated stent. The method includes sliding a proximal end of a sheath including at least one reduced thickness region over a distal end of the drug coated stent and contacting a tapered distal end of the sheath with the catheter. The method also includes flexing the sheath about the reduced thickness regions while inserting the catheter mounted drug coated stent into an arcuate guide of a transport package.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiments of the present invention are illustrated by the accompanying figures, wherein:

FIG. 1 is an illustration of a sheath, in accordance with a first embodiment of the current invention;

FIG. 2 is an illustration of the sheath of FIG. 1, in a flexed position;

FIG. 3 is an illustration of a prior art drug coated stent;

FIG. 4 is an illustration of a drug coated stent inside the sheath of FIG. 1;

FIG. 5 is an illustration of a drug coated stent flexed inside the flexed sheath of FIG. 2;

FIG. 6 is an illustration of a stent delivery catheter inserted to different positions within an arcuate catheter guide;

FIG. 7 is an illustration of a transport package;

FIG. 8 is an illustration of a sheath, in accordance with a second embodiment of the current invention;

FIG. 9 is an illustration of a sheath, in accordance with a third embodiment of the current invention;

FIG. 10 is an illustration of a sheath, in accordance with a fourth embodiment of the current invention;

FIG. 11 is an illustration of a stylet inserted into the sheath of FIG. 1; and

FIG. 12 is an illustration of a sheath, in accordance with a fifth embodiment of the current invention.

### DETAILED DESCRIPTION OF THE PRESENTLY PREFERRED EMBODIMENTS

The invention is based on the observation that the drug on drug coated stents was removed by varying amounts from the stent by friction between the drug coated stent and the shipping hoop in a packaging system used to ship the drug coated stents to medical practitioners. When the drug is removed by varying amounts the quality of care to a patient is degraded by varying and unknown amounts. Thus in accordance with the invention, a sheath prevents friction on a drug coated stent when the stent attached to a catheter is threaded into the shipping hoop. In this manner the sheath ensures that all the drug on a drug coated stent remains on the stent after shipment to a medical professional.

The following description should be read with reference to the drawings in which like elements in different drawings are numbered identically. The drawings, which are not necessarily to scale, are not intended to limit the scope of the invention.

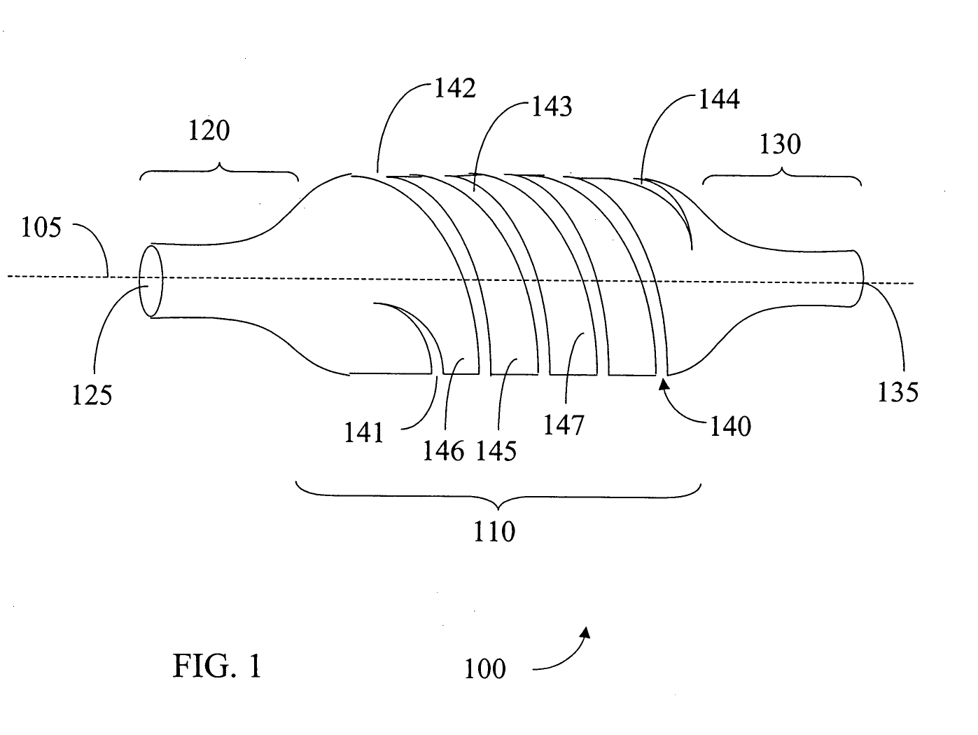

FIG. 1 shows one embodiment of a sheath 100. The body portion of sheath 100 has a sheath proximal end 120 with a proximal central lumen 125 and a sheath distal end 130, which tapers down from a main body portion 110 to a sheath distal closed region 135. The proximal end tapers up to the proximal end of the main body portion 110. The sheath distal closed region 135 and opposing proximal central lumen 125 are both centered upon the central axis 105 of the sheath 100.

The sheath 100 has a reduced thickness region forming a spiral configuration which encircles the main body portion 110 of the sheath 100. The reduced thickness region in this embodiment is a spiral cut 140. The spiral cut 140 begins at cut section 141 circles around the back side (not shown) of the main body portion 110 and is visible again at cut section 142. After a second pass around the back of the main body portion 110 the cut is seen at cut section 143. The cut sections 142 and 143 isolate the body section 145. Body sections 146 and 147 are on either side of body section 145 and they are all separated by spiral cut 140. The spiral cut 140 continues to diagonally encircle the main body portion 110 until it nears the end of main body portion 110 at cut section 144, where the spiral cut 140 ends. The diameter of the main body portion 110 is larger than the diameter of proximal central lumen 125 and the diameter of sheath distal closed region 135. The sheath 100 can be made out of plastics, such as polyethylene, polytrifluoroethlylene, polyurethane, polyelefin, vinyl derivatives, thermoplastics, thermosets, thermoplastic rubbers, and combinations thereof. The sheath 100 can be injected molded with the cuts 141, 142, 143, 144 or reduced thickness regions in place. In an alternate embodiment, sheath 100 is molded and then cuts are made in the main body portion 110. Reduced thickness areas may be used in place of cuts when the remaining material is flexible enough to stretch and buckle as required when the sheath 100 is flexed.

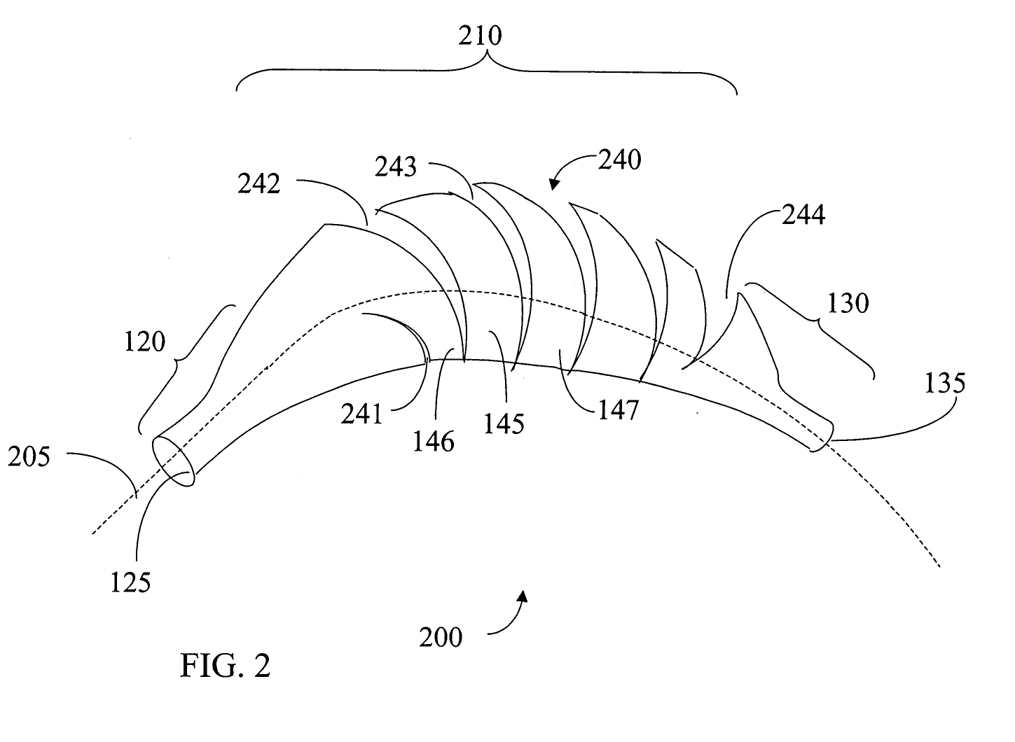

The spiral cut 140 permits the main body portion 110 to be flexed, as shown in FIG. 2, in which like elements share like reference numbers with FIG. 1. The flexed sheath 200 has a curved axis 205, which is centered in the proximal central lumen 125 and the sheath distal closed region 135 and has at least one radius of curvature. The spiral cut 140 inserted in the sheath 100 of FIG. 1 changes shape to that of cut 240 when the sheath 100 is flexed to form flexed sheath 200. The cut sections 242, 243 and 244 are now wider than cuts 142, 143 and 144 of FIG. 1 on the side opposite the radius of curvature of curved axis 205. At the top of the main body portion 210 the flexing has separated the edges of the cuts 242, 243 and 244. Additionally, the cut sections 241, 242 and 243 are narrower than cut sections 141, 142 and 143 of FIG. 1 on the side towards the radius of curvature of curved central axis 205. At the bottom of the main body portion 210 the flexing has brought the edges of the cuts 241, 242 and 243 close together. On the inside of the radius of curvature of curved central axis 205 the body section 145 touches adjacent body section 146 on its proximal edge and body section 145 touches adjacent body section 147 on its distal edge. On the outside of the radius of curvature of curved central axis 205 the body section 145 does not touch either body section 146 or body section 147.

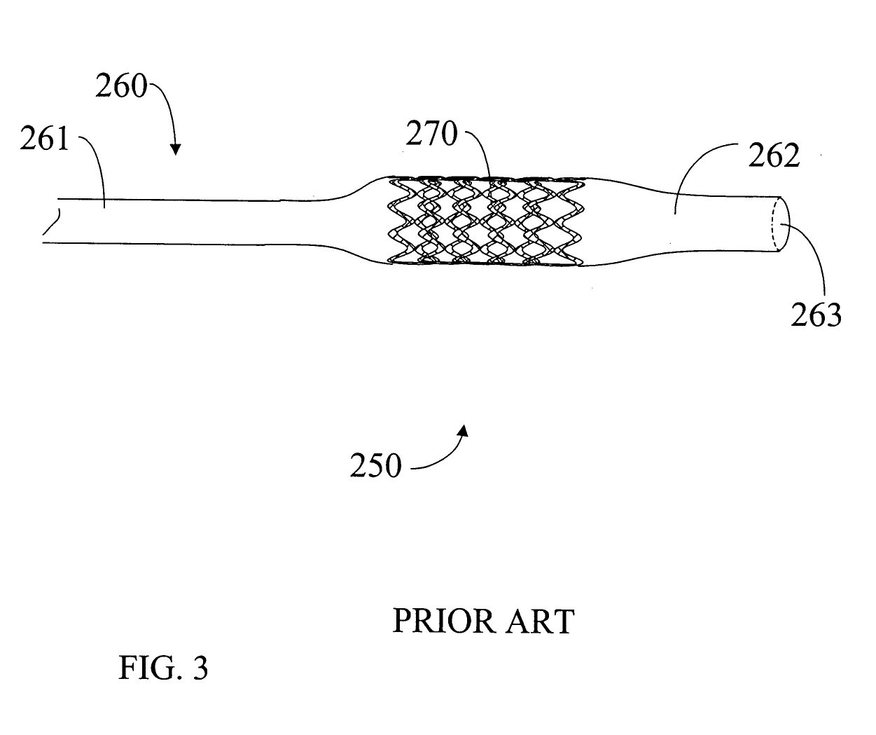

FIG. 3 shows a stent delivery assembly 250 to be inserted into sheath 100 for shipping to a medical practitioner. The main components of the stent delivery system 250 include an undeployed stent 270 and a stent delivery catheter 260 including a catheter proximal end 261 and a catheter distal end 262 and an central lumen 263, which is a tubular component. The use of stents and stent delivery systems is well known in the art. The stent may be deployed once in position within a lesion by expanding a balloon (not shown) folded under the stent 170 or by retracting a stent covering (not shown) that allows expansion of a self expanding stent. Once deployed the drug on the stent 270 is eluted to aid in the healing process.

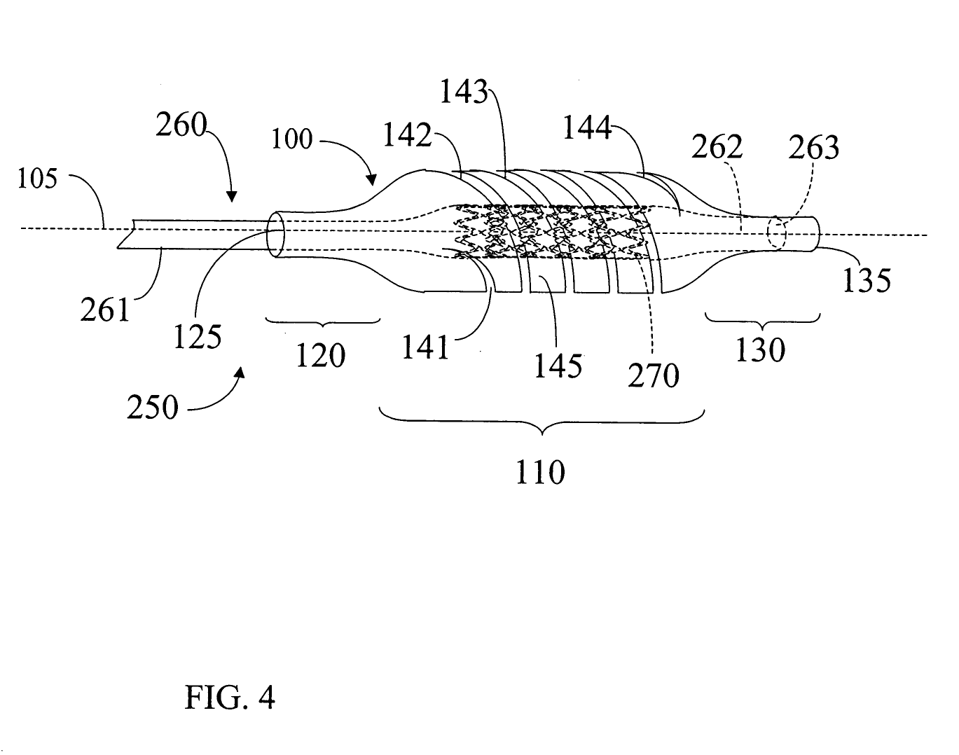

FIG. 4 shows a stent delivery assembly 250 after it has been inserted into a sheath 100. The outer diameter of the catheter distal end 262 is slightly larger than or just equal to the inner diameter of the sheath distal end 130 to provide an interference fit for the distal end 262 of the stent delivery catheter 260. The stent 270 is axially aligned with the central axis 105 within the main body portion 110 of the sheath 100. The catheter proximal end 261 is held within the opening 125 of the sheath 100. The diameter of the main body portion 110 is larger than the diameter of the sheath proximal end 120 and the diameter of sheath distal end 130. The diameter of the main body portion 110 is also larger than the undeployed stent 270 thus the undeployed stent 270 is not contacting any surface. The largest diameter of the stent delivery assembly 250 is smaller than the proximal central lumen 125 of the stent 100 to allow the proximal end of the sheath to slide over the distal portion of the stent delivery assembly 250.

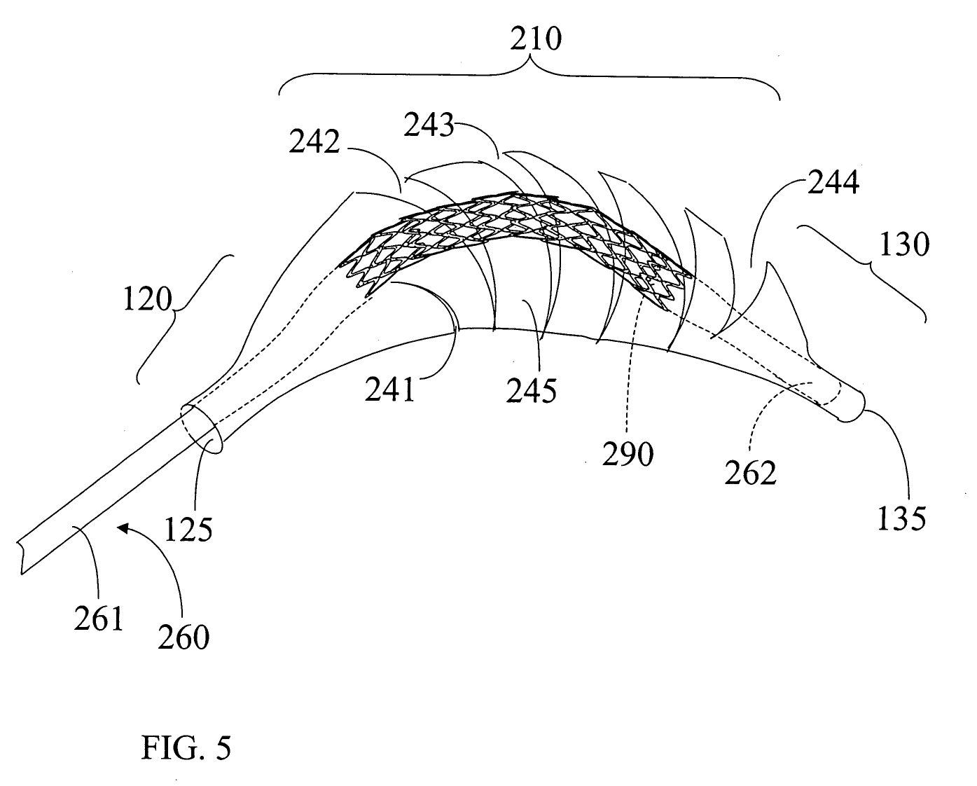

FIG. 5 shows the stent delivery assembly 250 after it has been inserted into a sheath 100 and flexed. The catheter distal end 262 of the flexed stent delivery catheter 260 maintains the interference fit in the sheath distal end 130 as stent delivery catheter 260 is flexed within flexed sheath 200. The catheter distal end 262 and the catheter proximal end 261 of the stent delivery catheter 260 hold the flexed undeployed stent 290 in a position which prevents the stent 270 from contacting any surface of the flexed sheath 200.

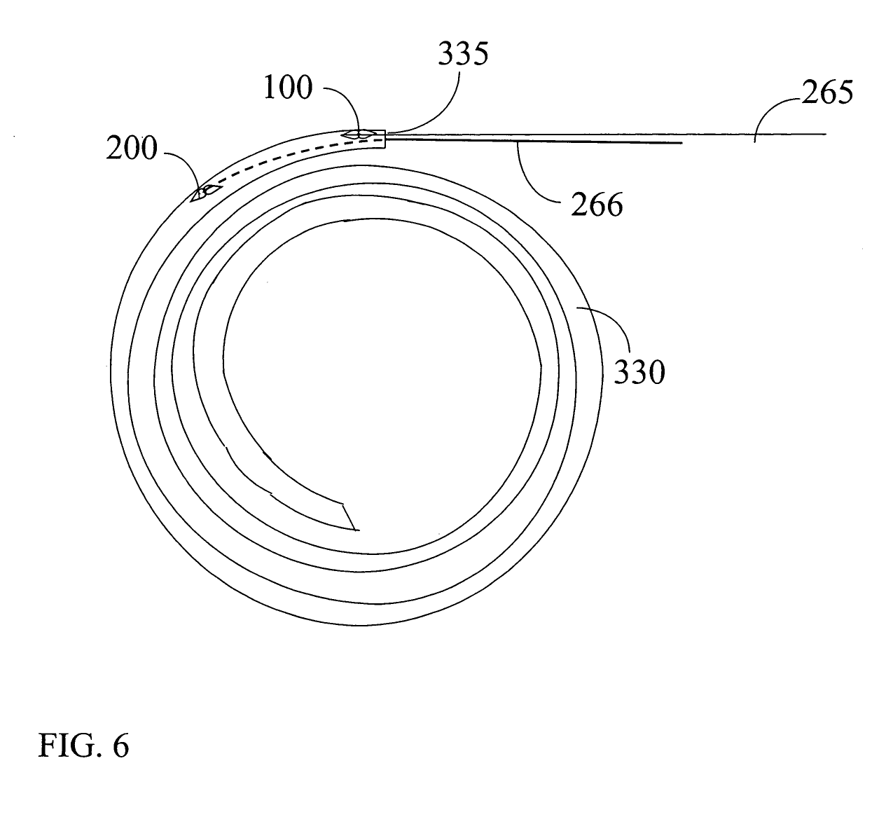

FIG. 6 is an illustration of a stent delivery catheter 260 inserted to different positions within an arcuate catheter guide 330. As a stent delivery catheter 260 is threaded into the input opening 335 of the arcuate catheter guide 330 it is in a first position shown by first inserted stent delivery catheter 265. The sheath 100, which holds the undeployed stent 270, is unflexed in this first position, since the sheath 100 has just entered the tubing of the arcuate catheter guide 330. In this position the central axis 105 of sheath 100 is about tangential to arcuate catheter guide 330. As the stent delivery catheter 260 continues to be threaded into the tube of the arcuate catheter guide 330 the sheath 100 moves to a second position shown by second inserted stent delivery catheter 266. Since the sheath 100 is now forced against the curved inner surface of the arcuate catheter guide 330 and sheath 100 is now flexed into the position of flexed sheath 200. If the flexed sheath 200 did not cover the stent 270 at least a portion of drug coated stent 270 would rub against the curved inner surface of the arcuate catheter guide 330. Some of the drug on the drug coated stent 270 would be left on the inner surface of the arcuate catheter guide 330 due to the friction with drug coated stent 270.

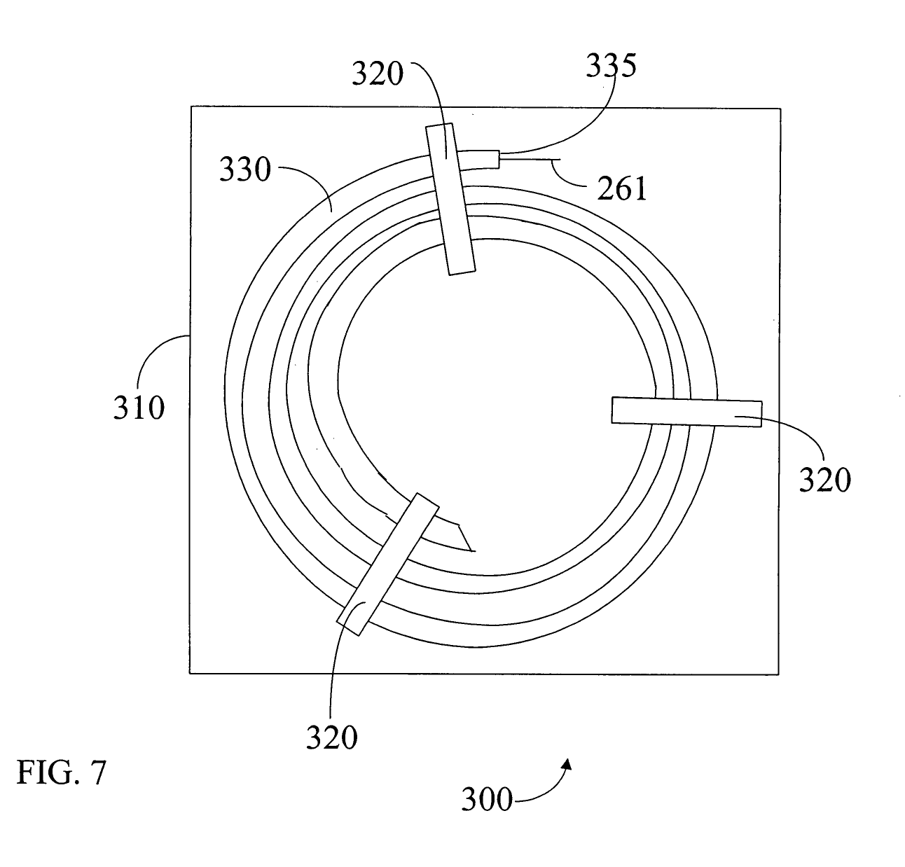

Once the stent delivery catheter 260 is completely threaded into the arcuate catheter guide 330 it placed in the shipping box 310, as shown in FIG. 7 where it is held in place with a plurality of attachment clips 320. Only a proximal portion of the catheter proximal end 261 of the stent delivery assembly 250 is visible outside the arcuate catheter guide 330. The sheath 200 will be in the flexed position while inside the arcuate catheter guide 330. If the transport package body 300 is shaken or dropped during shipping the stent 270 will remain untouched by any surface as it is securely held inside the main body portion 210 by the interference fit of the catheter distal end 262 within the sheath distal end 130.

The method for packaging a stent delivery catheter 260 mounted with a drug coated stent 270 begins by sliding proximal central lumen 125 of sheath 100 over the catheter distal end 262 of the stent delivery catheter 260. Then the catheter distal end 262 contacts the tapered sheath distal end 130 and is guided towards the sheath distal closed region 135. Next the central lumen 263 in centered within the sheath distal end 130 which holds catheter distal end 262 with an interference fit. The sheath 100 is flexed about the cuts 141, 142, 143 and 144 while the stent delivery catheter 260 mounted with drug coated stent 270 is inserted to the arcuate catheter guide 330 of a transport package body 300.

Once the transport package body 300 is delivered, the medical practitioner pulls the exposed end of the catheter proximal end 261 away from the input opening 335 of the arcuate catheter guide 330 to un-thread the stent delivery catheter 260. The flexed sheath 200 will continue to prevent friction between the drug coated stent 270 and the inner surface of the arcuate catheter guide 330 as the stent deliver catheter 260 is removed. The flexed sheath 200 will experience friction with the inner surface of the arcuate catheter guide 330 while protecting the stent 270. When the stent is to be delivered to the cardiovascular system or other vessels within the body of a patient the medical practitioner removes the sheath 100 by pulling the catheter proximal end 261 and the sheath distal closed region 135 in opposite directions.

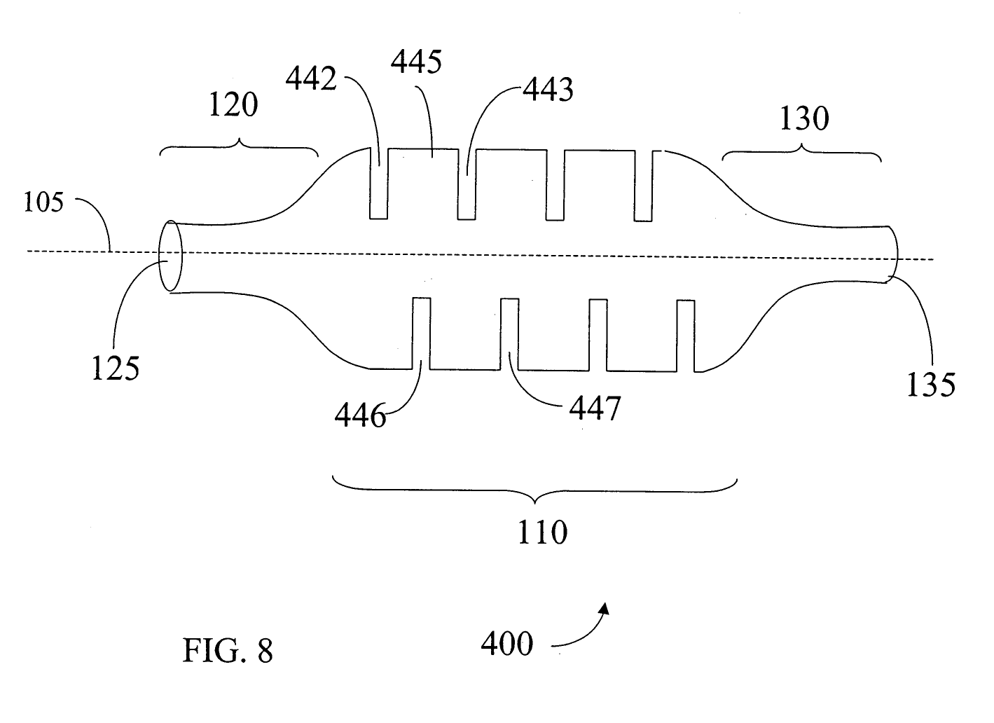

FIG. 8, in which like elements share like reference numbers with FIG. 1, shows a second embodiment of a sheath 400. Several circumferential cuts including 442, 443, 446, 447 provide the flexibility to sheath 400. The circumferential cut 446 is in between circumferential cuts 442 and 443 and on the opposite side of the main body portion 110 of the sheath 400. Main body section 445 is between circumferential cuts 442 and 443. The circumferential cuts including 442, 443, 446, 447 may extend the length of the main body portion 110. The spacing between cuts 442, 443, 446, 447 and the length and width of cuts 442, 443, 446, 447 may vary according the dimensions of the sheath 400.

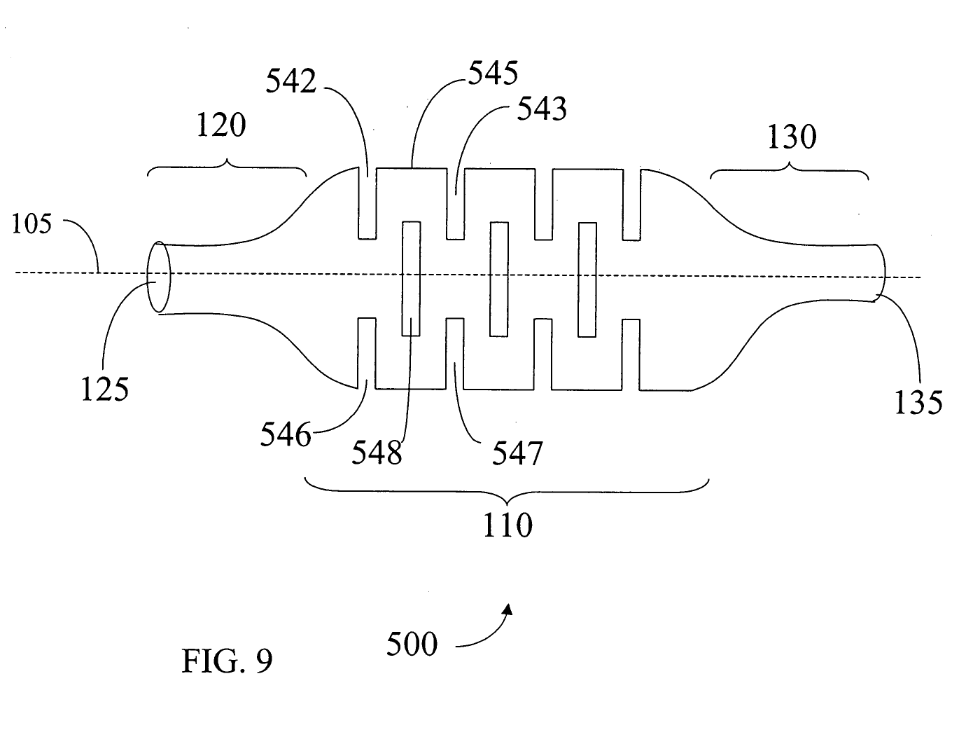

FIG. 9, in which like elements share like reference numbers with FIG. 1, shows a third embodiment of a sheath 500. Several circumferential cuts including 542, 543, 546, 547 and 548 provide the flexibility to sheath 500. The circumferential cut 542 is directly opposed to circumferential cut 546, which is on the opposite side of the main body portion 110 of the sheath 500. The circumferential cut 543 is directly opposed to circumferential cut 547, which is on the opposite side of the main body portion 110 of the sheath 500. Main body section 545 is between circumferential cuts 542 and 543. Circumferential cut 548 is disposed between the opposing pairs of cuts 542, 546, 545 and 547 and within a portion of the body section 545. The center of the arc of the cut 548 is about right angle with the center of the arc of the cuts 542, 543, 546 and 547. A cut directly opposing cut 548 and about the same length as cut 548 is on the back side of the main body portion 110. The circumferential cuts including 542, 543, 546, 547 and 548 extend the length of the main body portion 110.

The circumferential cuts including 542, 543, 546, 547 and 548 may extend the length of the main body portion 110. The spacing between cuts 542, 543, 546, 547 and 548 and the length and width of cuts 542, 543, 546, 547 and 548 may vary according the dimensions of the sheath 400.

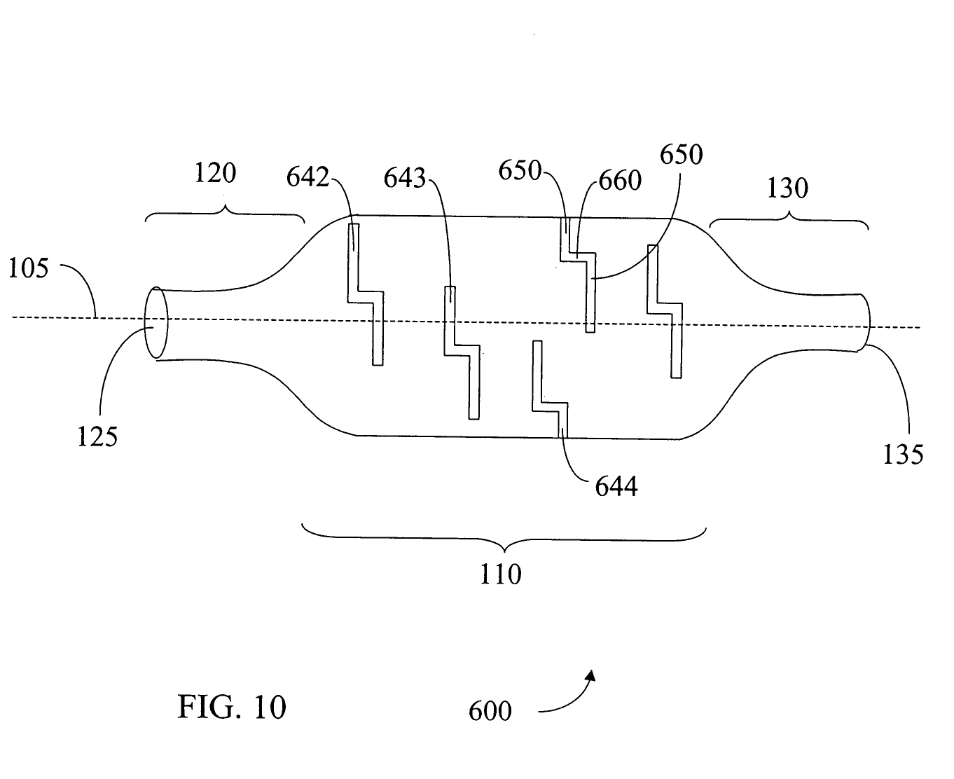

FIG. 10, in which like elements share like reference numbers with FIG. 1, shows a fourth embodiment of a sheath 600. Several staggered cuts including 642, 643 and 644 provide the flexibility to sheath 600. Each cut on sheath 600 comprises two circumferential cut sections 650 offset and connected by an axial cut section 660. Thus at least a portion of cut regions including 642, 643 and 644 are along at least a portion of a circumference of a main body portion 110 of the sheath 100. Axial cut section 660 is parallel the central axis 105 of the main body portion 110 of the sheath 100.

The cuts 642, 643 and 644 are located in a staggered relation to each other. For example, cut 643 is over and down from 642 while cut 644 is over and down from cut 643 and so forth. All the cuts including 642, 643 and 644 form a spiral pattern around the main body portion 110.

The cuts including 642, 643 and 644 may extend the length of the main body portion 110. The spacing between cuts 642, 643 and 644 and the length and width of cuts 642, 643 and 644 may vary according the dimensions of the sheath 600.

In an alternative embodiment, an alternating pattern of circumferential cut sections 650 and axial cut sections 660 may form one continuous cut spiraling around main body portion 110.

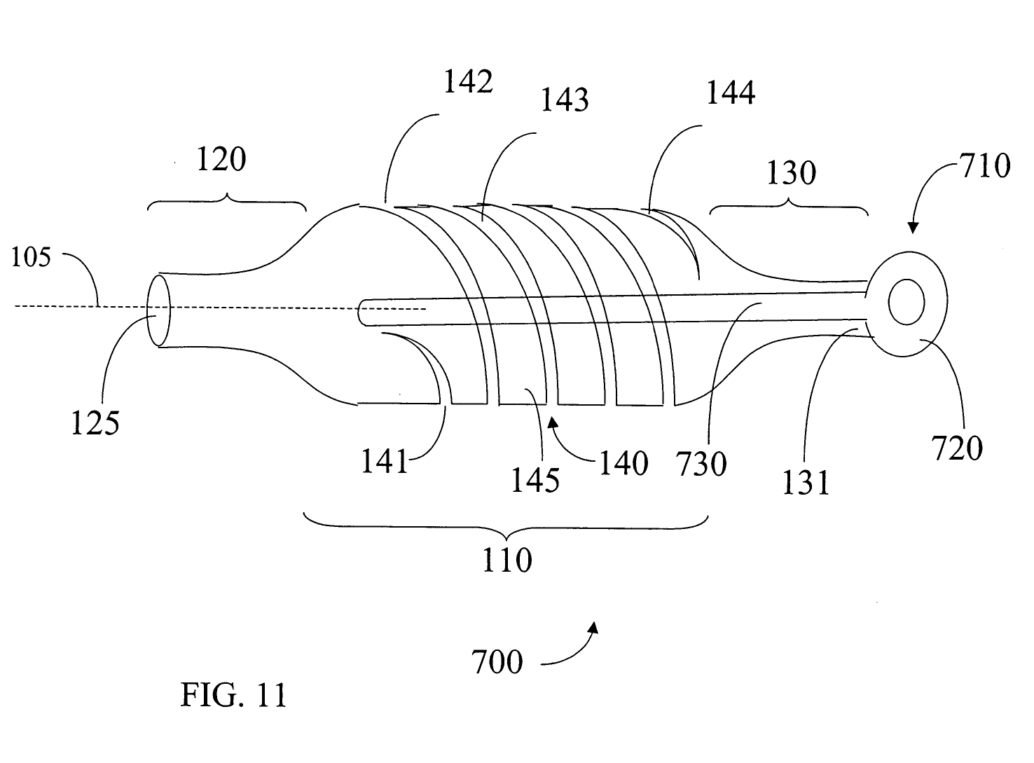

FIG. 11, in which like elements share like reference numbers with FIG. 4, shows how sheath 700 can accommodate a stylet 710, which comprises a stylet head 720 and a stylet pin 730. The stent delivery catheter 260 of FIG. 3 is not shown inserted in the sheath 700 in FIG. 11 for ease of viewing.

The function and use of stylets 710 is known in the art. A stylet pin 710 may be inserted into the central lumen 263 of a catheter distal end 262 during sterilization of the stent delivery catheter 260 to prevent the shrinkage of the inner diameter of the central lumen 263 in the catheter distal end 262. This is important if the catheter is designed with tight tolerances. Additionally, a stylet prevents kinking of the stent delivery catheter 260 around the stent 270 area, which may happen if the stent delivery catheter 260 is handled roughly when being removed from the arcuate catheter guide 330.

Stylets 710 are made from metal, such as stainless steel and plastics, such as polyethylene, polytrifluoroethlylene, polyurethane, polyelefin, vinyl derivatives, thermoplastics, thermosets, thermoplastic rubbers, and combinations thereof. Stylets come in various lengths dependant upon the stent length and may protrude from the protective sheath.

In FIG. 11, the stylet 710 is a separate piece from the sheath 700. The sheath distal closed region 135 of sheath 100 is replaced with a sheath distal opening 131 having a diameter larger than the diameter of the stylet pin 730 and smaller than the diameter of the stylet head 720. The inner diameter of the central lumen 263 of the catheter distal end 262 is larger than the outer diameter of the stylet pin 710. This fit provides the interference fit to hold the catheter distal end 262 in place in the sheath 700 when stylet 710 is inserted into the sheath 700 with the catheter distal end 262 positioned in the sheath distal opening 131. An interference fit is obtained by making the outer diameter of stylet pin 730 slightly larger than the inner diameter of central lumen 263. In an alternative embodiment, a rough surface finish on the stylet pin 730 provides the interference fit. In an alternative embodiment, a slight S curvature is added to the stylet pin 730 for the interference fit.

The method for packaging a stent delivery catheter 260 mounted with a drug coated stent 270 begins by sliding the proximal central lumen 125 of sheath 700 over the catheter distal end 262 of the stent delivery catheter 260. Then the catheter distal end 262 contacts the tapered sheath distal end 130 and is guided towards the sheath distal opening 131 so the central lumen 263 in centered within the sheath distal opening 131. Next the stylet pin 730 is inserted into the central lumen 263 of the catheter distal end 262. The sheath 700 is flexed about the cuts 141, 142, 143 and 144 while the stent delivery catheter 260 mounted with drug coated stent 270 is inserted to the arcuate catheter guide 330 of a transport package body 300.

In an alternative embodiment, the stylet 710 may be inserted into the sheath 700 and then the distal end of the stent delivery catheter 260 is inserted into the sheath 700. The tapering of the sheath distal end 130 guides the catheter distal end 262 towards the stylet pin 730. As the stylet pin 730 enters the central lumen 263 an interference fit is provided.

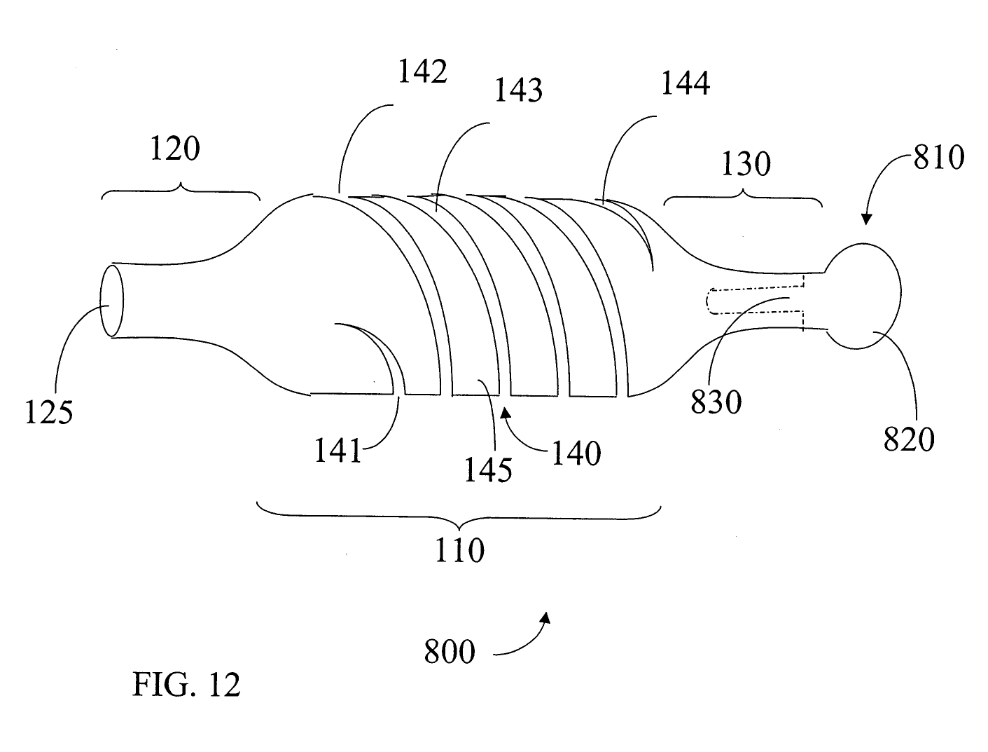

In FIG. 12, a stylet 810 is an integral piece with the sheath 800. The stent delivery catheter 260 of FIG. 3 is not shown inserted in the sheath 800 in FIG. 12 for ease of viewing. The sheath distal closed region 135 of sheath 100 in FIG. 4 is now shaped as a pin 830 and a tab 820. Tab 820 may have a shape similar to that of stylet head 720. The pin 830 has a diameter smaller than the inner diameter of the central lumen 263 of the catheter distal end 262. This fit provides the interference fit to hold the catheter distal end 262 in place in the sheath 800. Stylet pin 830 may have a diameter, which is slightly more than the inner diameter of central lumen 263. In this case, an interference fit to hold the catheter distal end 262 in place in the sheath 800 is provided. In an alternative embodiment, a rough surface finish on the stylet pin 730 provides the interference fit. In an alternative embodiment, a slight S curvature is added to the stylet pin 830 for the interference fit.

When the stent delivery catheter 260 is inserted into the sheath 800, catheter distal end 262 has an outer diameter smaller than the inner diameter of the sheath distal region 130. The central lumen 263 of the catheter distal end 262 will be positioned over pin 830 as the catheter distal end 262 is inserted into the sheath 800. The tapering of the sheath distal end 130 guides the inner lumen 263 over the pin 830.

The sheaths 100, 400, 500, 600, 700 and 800 can be made out of plastics, such as polyethylene, polytrifluoroethlylene, polyurethane, polyelefin, vinyl derivatives, thermoplastics, thermosets, thermoplastic rubbers, and combinations thereof. The sheaths 100, 400, 500, 600, 700 and 800 can be injected molded with the cuts or reduced thickness regions. In an alternative embodiment, sheaths 100, 400, 500, 600, 700 and 800 can be molded and then the main body portion 110 will be cut with the required pattern of cuts.

Reduced thickness regions may be used in place of some or all the cuts in the sheaths 100, 400, 500, 600, 700 and 800. For example, the cut sections 140, 141, 142, 143, 144 may be reduced thickness areas in sheath 100, which buckle and stretch as required when the sheath 100 is flexed. Various exemplary embodiments of cuts are presented but any cut or thickness reduction with allows for the main body portion 110 to flex without touching a stent 270 held within the sheaths 100, 400, 500, 600 and 700 is within the scope of the invention.

The stent delivery assembly 250 may be inserted in a flexible protective covering, such as a transparent plastic covering, to maintain sterility of the stent delivery assembly 250 prior to insertion into the arcuate catheter guide 330. In this case the sheaths 100, 400, 500, 600, 700 and 800 prevent the drug from being rubbed off of the drug coated stent 270 into the flexible protective covering.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

1. A system for packaging a drug coated stent, comprising:
a transport package body including an arcuate catheter guide;
a catheter including the drug coated stent disposed thereon and received in the catheter guide; and
a sheath disposed on the catheter and enclosing the drug coated stent, the sheath including at least one reduced thickness region therein to increase flexibility of the sheath.

2. The system of claim 1, further comprising a stylet inserted in a central lumen of the catheter and attached to a distal end of the sheath.

3. The system of claim 2, wherein the stylet and sheath are formed as a unitary member.

4. The system of claim 2, wherein a surface of a pin of the stylet is roughened to provide an interference fit.

5. The system of claim 2, wherein a pin of the stylet is formed in an S curve configuration to provide an interference fit.

6. The system of claim 1, wherein a distal end of the sheath is tapered to provide interference fit with the catheter.

7. The system of claim 1, wherein a distal end of the sheath is tapered to provide an interference fit with a stylet attached to a catheter.

8. The system of claim 1, wherein the reduced thickness region comprises at least one cut through the sheath.

9. A sheath for protecting a drug coated stent, comprising:
a main body portion including a central lumen for receiving a drug coated stent, the main body portion including at least one reduced thickness regions therein to increase flexibility of the sheath.

10. The sheath of claim 9, further comprising:
a distal end of the main body portion tapered to provide an interference fit with a portion of the catheter adjacent a distal end of the drug-coated stent.

11. The sheath of claim 9, wherein the main body portion has a spiral configuration, and the reduced thickness region comprises at least one cut which forms a spiral configuration.

12. The sheath of claim 9, wherein the reduced thickness region comprises a plurality of cuts.

13. The sheath of claim 11, wherein the plurality of cuts are parallel and spaced apart along the main body portion.

14. The sheath of claim 9 wherein the sheath is formed from a material selected from the group consisting of polyethylene, polytrifluoroethlylene, polyurethane, polyelefin, vinyl derivatives, thermoplastics, thermosets, thermoplastic rubbers, and combinations thereof.

15. A method for packaging a catheter mounted drug coated stent, the method comprising:
sliding a proximal end of a sheath including at least one reduced thickness region over a distal end of the drug coated stent;
contacting a tapered distal end of the sheath with the catheter; and
flexing the sheath about the reduced thickness regions while inserting the catheter mounted drug coated stent into an arcuate guide of a transport package.

16. The method of claim 15, wherein the tapered distal end of the sheath is centered about a central axis of the sheath.

17. The method of claim 15, wherein the contacting a tapered distal end of the sheath with the catheter comprises achieving an interference fit between the tapered distal end of the sheath and an outer surface of the catheter.

18. The method of claim 15, wherein the contacting a tapered distal end of the sheath with the catheter comprises inserting a stylet mounted on the distal end of the sheath through a central lumen of the catheter.

19. The method of claim 15, wherein flexing the sheath about the reduced thickness regions comprises flexing the sheath about cut regions in the sheath.

20. The method of claim 19, wherein the cut regions are in a spiral configuration about a main body portion of the sheath.

21. The method of claim 19, wherein the at least a portion of cut regions are along at least a portion of a circumference of a main body portion of the sheath.

22. The method of claim 21, wherein the at least a portion of cut regions are parallel an axis of a main body portion of the sheath.
